(19) United States
(12) Patent Application Publication
(10) Pub. No.: US 2025/0352208 A1
(43) Pub. Date: Nov. 20, 2025

(54) IMPLANTABLE ADJUNCT HAVING A FILM ATTACHMENT

(71) Applicant: CILAG GMBH INTERNATIONAL, ZUG (DE)

(72) Inventors: LAURA S. DOWNING, CINCINNATI, OH (US); MARK ZEINER, LOVELAND, OH (US)

(73) Assignee: CILAG GMBH INTERNATIONAL, ZUG (DE)

(21) Appl. No.: 19/093,403

(22) Filed: Mar. 28, 2025

Related U.S. Application Data
(60) Provisional application No. 63/649,611, filed on May 20, 2024.

(51) Int. Cl. A61B 17/072; A61B 17/00

(52) U.S. Cl. CPC A61B 17/07292; A61B 2017/00004; A61B 2017/00477; A61B 2017/00526; A61B 2017/07242

(57) ABSTRACT

Systems, apparatuses, and methods for attaching film layers to implantable adjuncts are disclosed.

### CROSS REFERENCE TO RELATED APPLICATION

This application claims the benefit of, and priority to, U.S. Provisional Application No. 63/649,611 filed on May 20, 2024, the disclosure of which is expressly incorporated herein by reference.

### FIELD OF INVENTION

The present disclosure generally relates to implantable adjuncts for surgical staplers. More specifically, the present disclosure relates to attaching a film layer to implantable adjuncts for surgical staplers.

### BACKGROUND

Stapling is a crucial aspect of many surgical procedures, such as gastrointestinal, thoracic, and gynecological surgeries. A pivotal aspect of a stapling procedure is to provide proper staple formation (i.e., the legs curling around at a proper position to secure tissue within the formed staple). Issues can occur, therefore, if conditions exist that take away from the possibility of proper tissue fastening. One such condition is when there is variation in tissue thickness across the length of the stapling surface.

Certain staple cartridges used in stapling procedures may include an implantable adjunct on the deck of the cartridge or on the anvil side of the device. This implantable adjunct can be used to compensate for differences in tissue thickness. For instance, tissue may be thicker at one end of a staple cartridge than at another, yet the length of staple legs is the same for all staples in the cartridge. As such, a staple can have proper length in one section of that tissue yet be too long for another section of the tissue. The implantable adjunct is stapled to the tissue, thereby compensating for some of the thickness variation, and, in some implementations, the implantable adjunct will biodegrade over time.

Since the adjunct is stapled to the tissue, the implantable adjunct must be constructed such that crowns of the staples do not pull through the adjunct-if staples pull through, then the tissue thickness is no longer compensated for. Various designs have been implemented to decrease the chance of staple pull-through. One such method is to include a film layer on the adjunct such that the adjunct comprises multiple layers, including for instance a foam or other porous material that is then laminated with a layer of film material. The multiple layers can include several layers of film and fabric located on either side of the device. In prior implementations, the film layer is heat bonded to the porous layer.

During the heat bonding process, the film material is melted onto the implantable adjunct to cause the film to adhere to the adjunct. Several issues can arise from this heat lamination. For instance, the application of heat during the lamination process can result in the formation of air pockets within the cushion that impact the structural integrity of the implantable adjunct. Heat lamination can also affect the thickness of the implantable adjunct.

There is a need for providing an implantable adjunct that includes one or more film attachments and methods for assembling such an implantable adjunct that maintain the structural integrity and performance of the implantable adjunct.

### SUMMARY

It is an object of the present designs to provide devices and methods to meet the above-stated needs. The designs can be for an implantable adjunct having one or more film layers mechanically attached thereto using a connector. It is to be understood that the one or more film layers may include various materials, including by way of example, mesh materials or knit fabrics, that are applied to the implantable adjunct. The implantable adjunct is formed without the need for heat bonding, which eliminates impacts to the structural integrity of the implantable adjunct that occur during heat lamination and provides improved performance for the implantable adjunct.

Other aspects of the present disclosure will become apparent upon reviewing the following detailed description in conjunction with the accompanying figures. Additional features or manufacturing and use steps can be included as would be appreciated and understood by a person of ordinary skill in the art.

### BRIEF DESCRIPTION OF THE DRAWINGS

The above and further aspects of this invention are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation. It is expected that those of skill in the art can conceive of and combine elements from multiple figures to better suit the needs of the user.

FIG. 1 is a perspective view of a replaceable staple cartridge without an adjunct.

FIG. 2A is a schematic view showing a replaceable staple cartridge, an attachment material, and an implantable adjunct.

FIG. 2B is a schematic view showing the replaceable staple cartridge, attachment material, and implantable adjunct of FIG. 2A assembled.

FIG. 2C is a perspective view showing the replaceable staple cartridge and implantable adjunct of FIG. 2A assembled.

FIG. 3A is a side-view schematic of a staple cartridge being loaded into a surgical instrument, according to aspects of the present disclosure.

FIG. 3B is a schematic view of an implantable adjunct stapled to tissue, according to aspects of the present disclosure.

FIG. 4 is a perspective, exploded view of an implantable adjunct with two film layers, according to aspects of the present disclosure.

FIG. 5 is a perspective of an implantable adjunct with a film layer attached via connectors, according to aspects of the present disclosure.

FIG. 6A is a transparent side view of an implantable adjunct with a film layer attached via connectors, according to aspects of the present disclosure.

FIG. 6B is an enlarged view of the area highlighted in FIG. 6A, according to aspects of the present disclosure.

FIG. 7 is a perspective of an implantable adjunct with a film layer attached via connectors, the connectors providing forces at the edges of the adjunct, according to aspects of the present disclosure.

FIG. 8 shows an example double needle.

FIG. 9 is a flowchart showing an example method for assembling an implantable adjunct, according to aspects of the present disclosure.

### DETAILED DESCRIPTION

Specific examples of the present invention are now described in detail with reference to the Figures, where identical reference numbers indicate elements which are functionally similar or identical. The examples provide solutions for staple cartridge systems that include an implantable adjunct. An implantable adjunct can be used in stapling surgery to account for differing tissue thicknesses across the length of the stapling surface. For instance, a length of tissue clamped in an end effector of a surgical instrument may be thicker at one end of the staple cartridge than at the other end. However, the staple cartridge may be loaded with staples of a single length, meaning the staples may be properly sized for the thicker section of tissue, but may be too long for the thinner section of tissue. If the staples are too long, proper compression of the tissue at the staple site may not be optimal. An implantable adjunct can account for this differing tissue thickness by providing support for the thinner sections of tissue. Where the tissue is thick, the implantable adjunct can be compressed all the way down since no additional thickness is needed to account for the staple length. Where the tissue is thin, the implantable adjunct is not as compressed, meaning the adjunct provides the additional thickness needed to account for the staple length, thereby providing proper compression in that section of the tissue.

The staple cartridge can also include an implantable adjunct. The implantable adjunct is configured to be captured within a staple along with tissue when the staple is deployed by the corresponding driver. The implantable adjunct can comprise a buttress, a tissue thickness compensator, and/or other adjunct material. A tissue thickness compensator is configured to compensate for variations in tissue properties, such as variations in the thickness of tissue, for example, along a staple line. A tissue thickness compensator can be compressible and resilient. In use, a tissue thickness compensator prevents or limits the over-compression of stapled tissue while facilitating adequate tissue compression within and between staples.

The implantable adjunct of a staple cartridge can be releasably secured to the body of the staple cartridge. For example, the implantable adjunct can be releasably secured to the deck of the staple cartridge with a releasable adhesive, at least one attachment tab, and/or other attachment features.

As discussed above, the implantable adjunct accounts for differing tissue thickness by providing support for the thinner sections of tissue. In that regard, implantable adjunct may include a foam or cushion material that allows for compression. The foam can include one or more film layers attached thereto using a connector, such as a thread. For example, a film layer may be applied to allow the implantable adjunct to more easily slide over tissue by providing a smoother surface than the underlying foam. In other examples, a film layer may be applied to facilitate attachment of the implantable adjunct to the staple deck. It is to be understood that the term film layers could include materials such as meshes or knit fabrics or other materials, such as therapeutics. The attachment of film layers using a connector, such as a thread, eliminates damage to the layers that would result from heat bonding to provide additional strength to the implantable layer and improve staple pull through during operation. Further, the applied film layers do not have to be melted and therefore, thinner films can be employed.

The invention is not necessarily limited to the examples described, which can be varied in construction and detail. The terms “distal” and “proximal” are used throughout this description and are meant to refer to positions and directions relative to the handle of surgical instrument 200. As such, “distal” or distally” refer to a position distant to or a direction away from the handle of surgical instrument 200 (i.e., a direction toward a patient). Similarly, “proximal” or “proximally” refer to a position near or a direction towards the handle of surgical instrument 200 (i.e., toward an operator of the handle). Furthermore, the singular forms “a,” “an,” and “the” include plural referents unless the context clearly dictates otherwise. Furthermore, the use of “couple”, “coupled”, or similar phrases should not be construed as being limited to a certain number of components or a particular order of components unless the context clearly dictates otherwise.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 109%.

The components described herein can be formed from biocompatible materials using manufacturing methods known to those of skill in the art. For example, and not limitation, the components described herein can be formed of a thermoset (e.g., the adjunct) or molded from a thermoplastic (e.g., the film or knit layers).

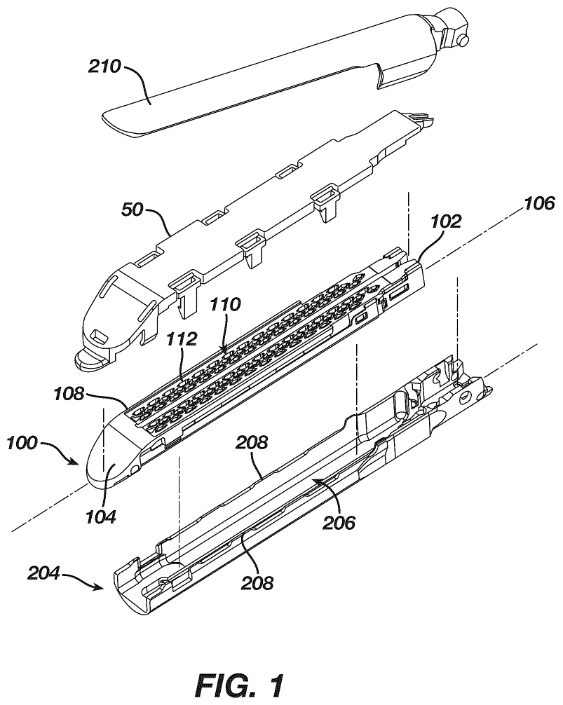

FIG. 1 provides background on how the presently described adjuncts and systems interact with a staple cartridge. FIG. 1 shows an exploded view of a staple cartridge 100 that does not include an implantable adjunct on deck 108 thereof. In these prior examples, retainer 50 can be attached to staple cartridge 100 from proximal end 102 to distal end 104 to ensure that staples within various staple pockets 110 do not fall out of openings 112 within deck 108. Retainer 50, therefore, is a static device with a function of preventing staples from falling out before staple cartridge 100 is positioned within channel 206 of first jaw frame 204 of end effector 202. Retainer 50 can be removed when staple cartridge 100 is inserted between channel rails 208 of channel 206.

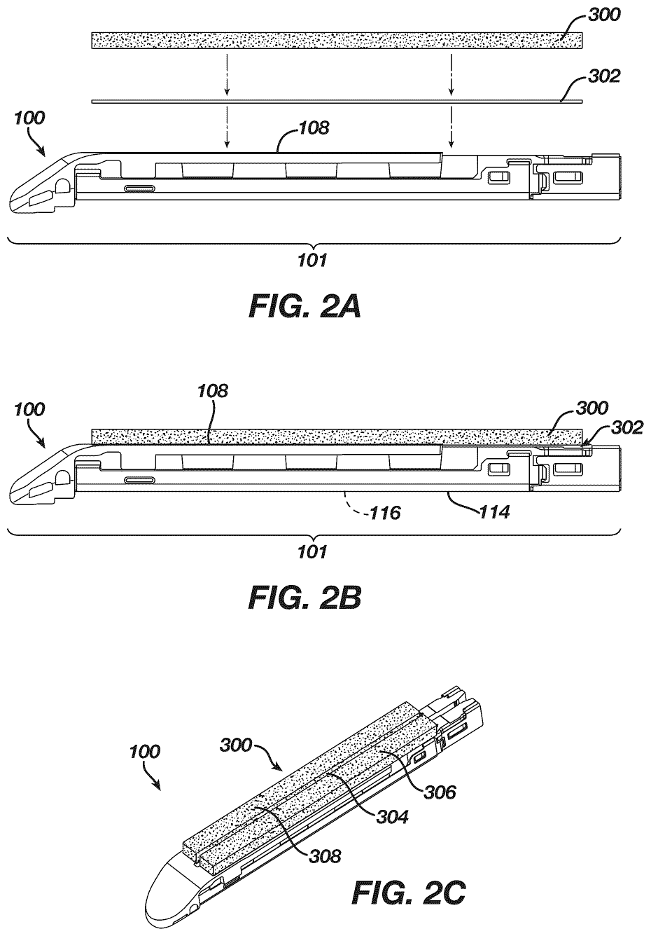

FIGS. 2A-2C illustrate staple cartridges that include an implantable adjunct 300. As seen in the exploded view of FIG. 2A, a system benefiting from the presently disclosed designs includes staple cartridge 100 (which is substantially similar to the staple cartridge shown in FIG. 1) and implantable adjunct 300 which is adhered to deck 108 that is positioned along elongate body 101 of staple cartridge 100. Implantable adjunct 300 can be adhered to staple cartridge 100 with attachment material 302. Attachment material 302 can provide sufficient adhesion for implantable adjunct 300 to remain adhered to deck 108 when being positioned at the treatment site, but the adhesion does not impair the ability of implantable adjunct 300 from being detached from deck 108 when being implanted. In some instances, attachment material 302 can be an adhesive, adhesive strip, double-sided tape, and the like. The attachment material 302 can be or include a pressure sensitive adhesive such that applying pressure to implantable adjunct 300 causes the attachment material 302 to be compressed and increases the adhesion of the attachment material 302 to the cartridge 100 and the implantable adjunct 300. In other words, compression of the adjunct 300 against the cartridge 100 can increase the adhesion of the implantable adjunct 300 to the cartridge 100 via the attachment material 302. The compression of the attachment material 302 can be less than a second and greatly increases the adhesion of the adjunct 300 to the cartridge.

FIG. 2B shows implantable adjunct 300 adhered to deck 108 via attachment material 302. FIG. 2C is a perspective view of implantable adjunct 300 adhered to deck 108. For background, the staples of the systems described herein are fired through implantable adjunct 300 during the stapling procedure. In some instances, implantable adjunct 300 can include sled groove 304 along the longitudinal axis 350 of the adjunct. Sled groove 304 provides a path for a knife (not shown in figures) to traverse such that the knife does not need to cut through adjunct 300, thereby preserving the edge on the knife. When implantable adjunct 300 includes sled groove 304, implantable adjunct 300 can be considered to be separated into adjunct first side 306 and adjunct second side 308. In some examples, implantable adjunct 300 can include laminated layers, such as a foam and/or porous material laminated with a mesh material, wherein the sled groove 304 is disposed in the foam and/or porous material but the mesh material remains intact. In other examples, implantable adjunct 300 can include a film layer and/or a mesh layer and/or a knit fabric layer, as will be described in greater detail below.

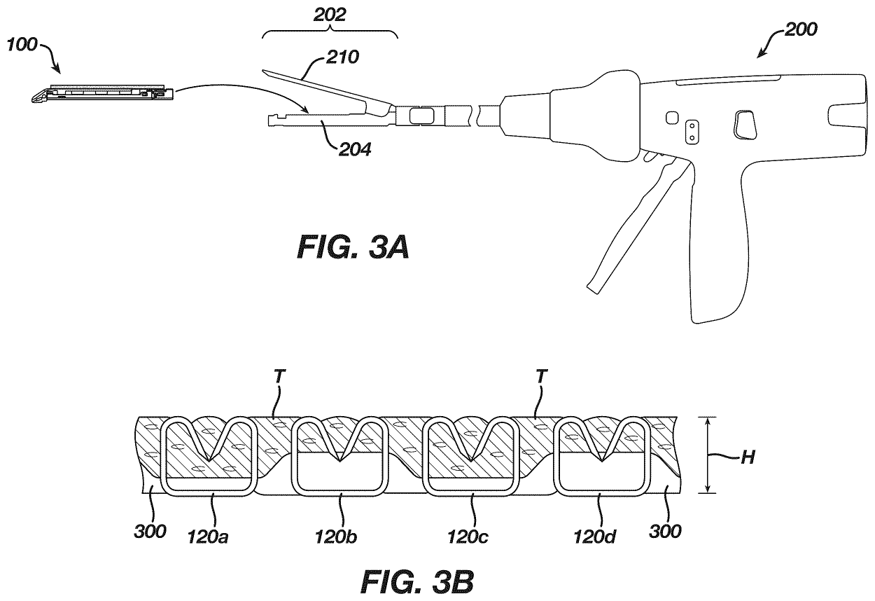

FIG. 3A is a side-view schematic of staple cartridge 100 being loaded into a surgical instrument, i.e., surgical instrument 200. Staple cartridge 100 is loaded into end effector 202 before being positioned at the treatment site. As described above, staple cartridge 100 is inserted into first jaw frame 204. Anvil 210 clamps down toward staple cartridge 100 during the stapling procedure. Once the tissue is stapled, anvil 210 opens to leave the staples and implantable adjunct 300 attached to the tissue. Staple cartridge 100 remains in first jaw frame 204 as surgical instrument 200 is removed from the treatment site. Although FIG. 3A shows staple cartridge 100 without a retainer attached thereto, some example retainers described herein can be configured to be inserted into first jaw frame 204 while attached to the staple cartridge 100. As is shown in FIG. 2B, cartridge 100 includes a pan 114 to support the elongate body 101, the bottom surface 117 of pan 114 being adjacent first jaw frame 204 when inserted into the first jaw frame 204 of end effector 202.

As stated above, implantable adjunct 300 can account for this differing tissue thickness by providing support for the thinner sections of tissue. Where the tissue is thick, implantable adjunct 300 can be compressed all the way down since no additional thickness is needed to account for the staple length. Where the tissue is thin, the implantable adjunct 300 is not as compressed, meaning the adjunct provides the additional thickness needed to account for the staple length, thereby providing proper compression in that section of the tissue. FIG. 3B is a schematic showing the implantable adjunct 300 stapled to tissue (T) having different thickness. The individual staples 120a,b,c,d have the same height (H), so the implantable adjunct 300 fills in the space for thinner sections of tissue (i.e., the tissue (T) shown at staples 120b and 120d). For thicker sections of tissue (i.e., the tissue (T) shown at staples 120a and 120c), the implantable adjunct 300 is more compressed as the staples do not need the additional space (i.e., height) filled in by implantable adjunct 300.

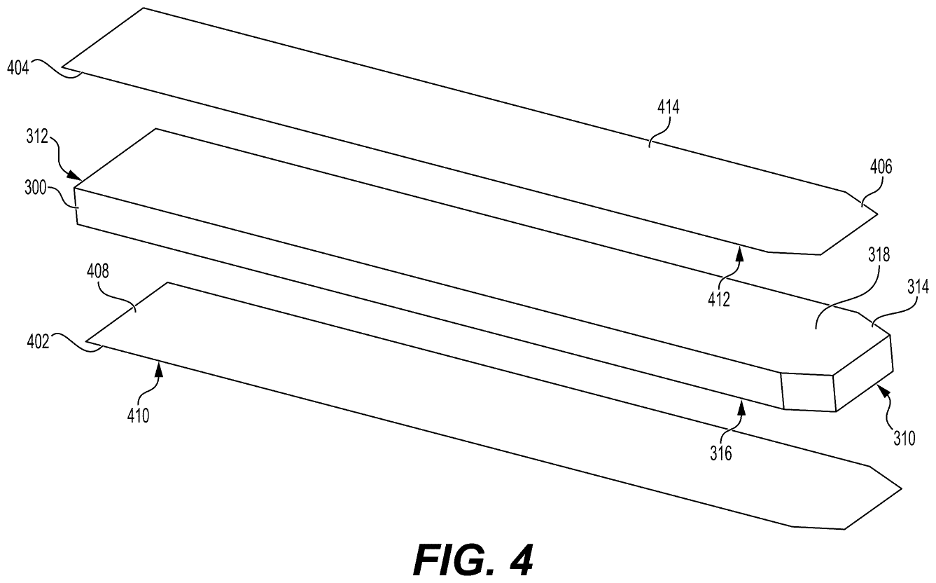

FIG. 4 illustrates an exploded view of an exemplary implantable adjunct 300 configured to be detachably adhered to deck 108 of staple cartridge 100. Implantable adjunct 300 can be employed in the system described above with respect to FIGS. 2A-2C, for example. Implantable adjunct 300 advantageously includes one or more film layers mechanically attached thereto. The mechanical attachment avoids problems associated with heat lamination and provides increased structural integrity and improved staple pull through during use.

Referring again to FIG. 4, in this example, implantable adjunct 300 is shown in the exploded view with a first film layer 402 and a second film layer 404 on opposing sides thereof. The film layers 402 and 404 are configured to be mechanically attached to the opposing sides of implantable adjunct 300, as describe in further detail below. Although first film layer 402 and second film layer 404 are both shown and described, it is to be understood that in some examples, either first film layer 402 or second film layer 404 could be utilized alone with implantable adjunct 300 without including the other film layer. First film layer 402 and second film layer 404 can be mechanically attached to implantable adjunct 300, as described in further detail below. The mechanical attachment prevents structural damage to implantable adjunct 300 from the application of the heat that would be required for heat lamination and provides for improved performance of implantable adjunct 300.

In this example, implantable adjunct 300 extends along a length between a proximal end 310 and a distal end 312. Implantable adjunct 300 includes a chamfer 314 at the proximal end 310, although implantable adjunct 300 may have other configurations. In some examples, implantable adjunct 300 can be a foam and/or porous material, such as a thermoset polymer, by way of example only. In additional examples, implantable adjunct 300 can include laminated layers, such as a foam and/or porous material laminated with a mesh material or a knit fabric. The laminated layers could include mesh, knit, film, non-woven, or other materials such as healing agents, or coagulating materials, by way of example only. Implantable adjunct 300 includes at least one layer formed of an absorbable material, as known in the art.

Implantable adjunct 300 includes a deck-facing surface 316 and an external surface 318 located on opposing sides thereof. Deck-facing surface 316 and external surface 318 are defined based on the orientation in which implantable adjunct 300 is to be adhered to deck of staple cartridge 100 and are otherwise not intended to be limiting. In some examples, implantable adjunct 300 has a thickness between deck-facing surface 316 and external surface 318 of less than about 3 millimeters. Implantable adjunct 300 including the first film layer 402 and the second film layer 404, as described below, can have a thickness of about 3 millimeters. As described above, implantable adjunct 300 can include other laminated layers, such as a mesh or knit material in addition to first film layer 402 and second film layer 404.

First film layer 402 includes a first adjunct facing side 408 and a first exterior side 410 on opposing sides thereof. First adjunct facing side 408 and first exterior side 410 are defined based on the orientation in which first film layer 402 is attached to implantable adjunct 300, as described below, and are otherwise not intended to be limiting. First adjunct facing side 408 contacts deck-facing surface 316 of implantable adjunct 300 when mechanically attached thereto, as described in further detail below, and first exterior side 410 faces away from implantable adjunct 300. In this example, first exterior side 410 can be adhered to deck 108 of staple cartridge 100, for example, using attachment material 302 positioned between first film layer 402 and deck 108, as described above. First film layer 402 can be a thin film layer formed of a thermoplastic material, although first film layer 402 can be formed of other materials. The film layers 402, 404 can be formed in whole or in part of a biodegradable polymer, including but not limited to polymers such as polydioxanone (PDO). In some examples, first film layer 402 has a thickness of about 20 microns, although other films having other thicknesses can be employed. The use of mechanical attachment, as described herein, allows for first film layer 402 to be thinner as first film layer 402 does not have to be melted onto implantable adjunct 300. First film layer 402 in some examples can be a layer that includes a discontinuous first exterior side 410, such as a mesh layer or a knit layer.

Second film layer 404 includes a second adjunct facing side 412 and a second exterior side 414 on opposing sides thereof. Second adjunct facing side 412 and second exterior side 414 are defined based on the orientation in which second film layer 404 is attached to implantable adjunct 300, as describe below, and are otherwise not intended to be limiting. Second adjunct facing side 412 contacts external surface 318 of implantable adjunct 300 when mechanically attached thereto, as described in further detail below, and second exterior side 414 faces away from implantable adjunct 300. Second exterior surface 414 provides a surface that creates less friction and allows implantable adjunct 300 to glide across tissue during use. In this example, second film layer 404 includes a film chamfer 406 that is configured to align to chamfer 314 of implantable adjunct, although second film layer 404 can have other configurations. Second film layer 404 can be a thin film layer formed of a thermoplastic material, such as polydioxanone (PDO), although second film layer 404 can be formed of other materials. In some examples, second film layer 404 has a thickness of about 20 microns, although other films having other thicknesses can be employed. The use of mechanical attachment, as described herein, allows for second film layer 404 to be thinner as second film layer 404 does not have to be melted onto implantable adjunct 300. Second film layer 404 in some examples can be a layer that includes a discontinuous second exterior side 414, such as a mesh layer or a knit layer.

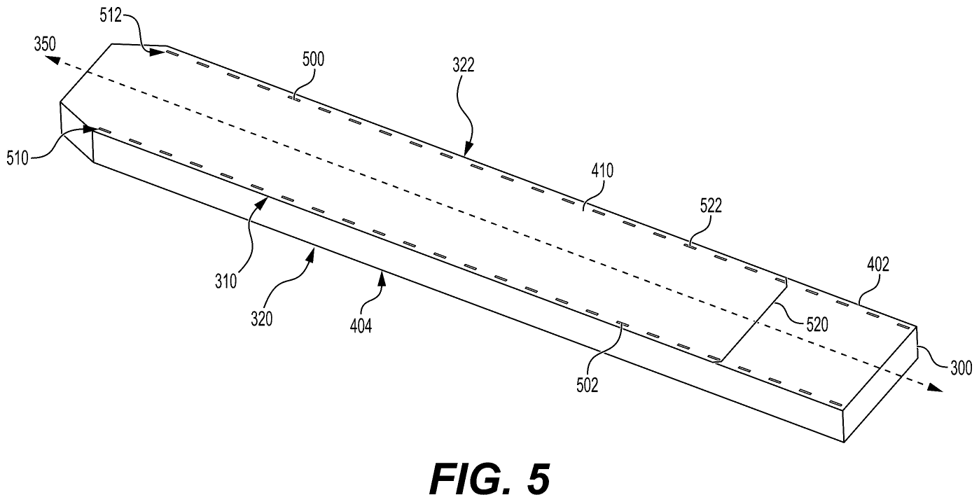

FIG. 5 is a perspective of an implantable adjunct 300 with a first film layer 402 and second film layer 404 attached thereto using a connector 500. It will be appreciated that the view in FIG. 5 is such that first film layer 402 is facing upward, and second film layer 404 is positioned below the implantable adjunct 300 such that second film layer 404 is hidden in the perspective view. Stated otherwise, FIG. 5 illustrates implantable adjunct 300 rotated 180 degrees and inverted as compared to the view shown in FIG. 4. Although first film layer 402 and second film layer 404 are both described with respect to FIG. 5, it is to be understood that in some examples, either first film layer 402 or second film layer 404 could be utilized alone with implantable adjunct 300 without including the other film layer.

As shown in FIG. 5, connector 500 is in contact with first exterior side 410 of first film layer 402 and provides a force on first exterior side 410 that attaches first film layer 402 to the deck-facing surface 316. Although not shown in FIG. 5, in some examples, connector 500 is also in contact with second exterior side 414 of second film layer 404 and provides a force on second exterior side 414 that attaches second film layer 404 to external surface 318 of implantable adjunct 300. Referring again to FIG. 5, in this example, connector 500 extends at least partially through the implantable adjunct 300 to attach first film layer 402. In some examples, connector 500 is a thread extending through the first film layer 402 and/or second film layer 404. In some embodiments, the thread comprises an absorbable material, including but not limited to bioabsorbable sutures known in the art. These bioabsorbable materials for the thread can include polydioxanone (PDO), polylactic acid (PLA), polyglycolic-acid, polycaprolactone (PCA), and the like or any combination thereof.

In this example, connector 500 includes a first row of stitching 510 positioned proximate a first edge 320 of the implantable adjunct 300. The connector 500 also includes a second row of stitching 512 positioned proximate a second edge 322. First row of stitching 510 and second row of stitching 512 are positioned inward of first edge 320 and second edge 322, respectively, to avoid pulling through the outside of implantable adjunct 300 during the insertion of connector 500. In some examples, first row of stitching 510 and second row of stitching 512 are located outside of the staple line when implantable adjunct 300 is adhered to deck 108 of cartridge 100.

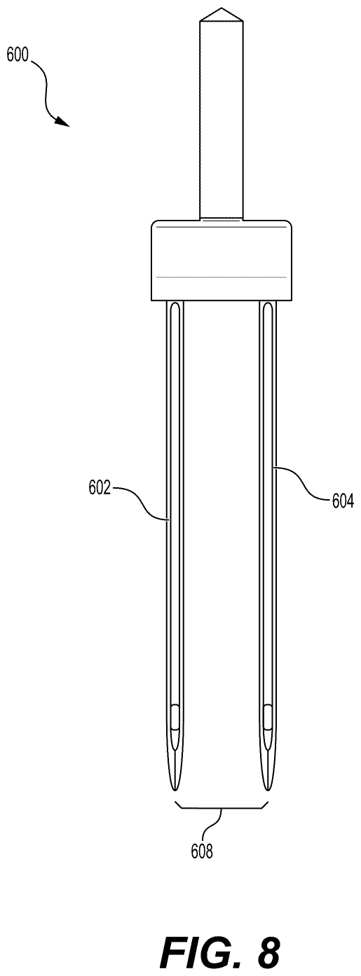

In this example, first row of stitching 510 and the second row of stitching 512 run parallel to each other to provide a consistent stich separation width 520. For example, first row of stitching 510 and the second row of stitching 512 can be inserted into implantable adjunct 300, as described in further detail below, using a double-needle sewing machine employing a double needle 600 as shown in FIG. 8, for example. The use of double needle 600 allows for aligning first row of stitching 510 and second row of stitching 512 to maintain stich separation width 520 along implantable adjunct 300.

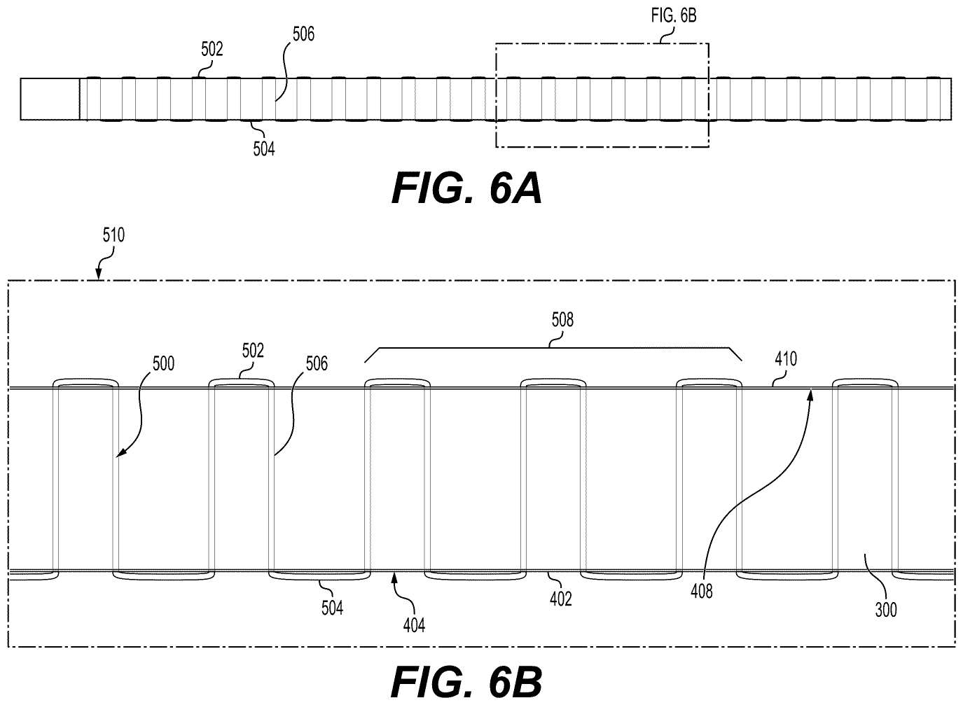

FIG. 6A is a transparent side view of an implantable adjunct 300 with a first film layer 402 and second film layer 404 attached thereto using a connector 500 and FIG. 6B is an enlarged view of the area of implantable adjunct 300 highlighted in FIG. 6A that illustrates a stitch pattern 508 for first row of stitching 510. Although stitch pattern 508 is illustrated and described for first row of stitching 510, it is to be understood that second row of stitching 512 (see, e.g., FIG. 5) can have a similar stitching pattern.

As shown in FIG. 6B, first row of stitching 510, which in this example is a thread, includes a plurality of first surface segments 502 that contact first exterior side 410 of first film layer 402. Although a simple stitch is illustrated, it is to be understood that other types of stitches known in the art could be utilized. For example, a lockstitch that uses upper and lower threads that entwine in the hole in the fabric which they pass through could be employed. The first surface segments 502 are shown in FIG. 6B as slightly separated from first exterior side 410 for illustration purposes only. In use, first surface segments 502 would be pulled against the first exterior side 410. First surface segments 502 provide the force to attach first film layer 402 to implantable adjunct 300. As shown in FIG. 5, second row of stitching 512 can similarly include second surface segments 522 that contact first exterior side 410 of first film layer 402 to provide the force on first exterior side 410 to attach first film layer 402 to implantable adjunct 300.

Referring again to FIG. 6B, first row of stitching further includes transverse segments 506 that are positioned at each side of each of the first surface segments 502 and extending at least partially through implantable adjunct 300. In some examples, as shown in FIG. 6B, transverse segments 506 extend through the entire thickness of implantable adjunct 300 to form a plurality of opposing surface segments 504 located on the opposing side of implantable adjunct 300. For example, opposing surface segments 504 are positioned to contact second exterior side 414 (see FIG. 4) of second film layer 404. The opposing surface segments 504 are shown in FIG. 6B as slightly separated from second exterior side 414 for illustration purposes only. In use, second surface segments 504 would be pulled against the second exterior side 414. In this example, opposing surface segments 504 can provide the force to attach second film layer 404 to implantable adjunct 300. In other examples, first film layer 402 and second film layer 404 are separately attached to implantable adjunct 300. In one example, stitch pattern 508 can include a backstitch at each end of first row of stitching 510.

As shown in FIG. 5, in this example, first surface segments 502 of first row of stitching 510 and second surface segments 522 of second row of stitching 512 are aligned laterally with respect to other each along a longitudinal axis 350 of implantable adjunct 300. For example, surface segments proximate one edge can be aligned with surface segments proximate the other edge. In some examples, second row of stitching 512 also includes transverse segments and opposing surface segments similar to the surface segments shown with respect to first row of stitching 510 in FIGS. 6A and 6B. This configuration can be obtained using double-needle 600 as illustrated in FIG. 8, as described in further detail below.

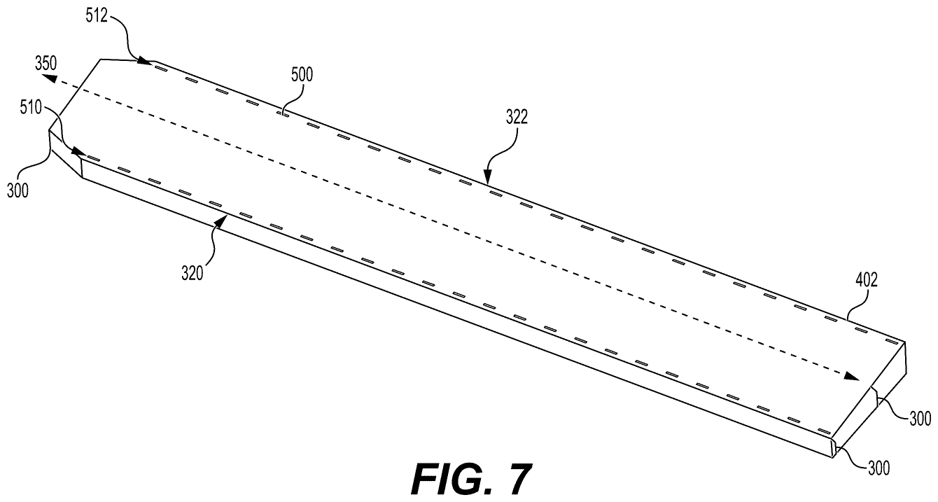

FIG. 7 is a perspective of an implantable adjunct 300 with film layer 402 attached via connectors, such as first row of stitching 510 and second row of stitching 512, although other types of connectors could be employed. In this example, first row of stitching 510 and second row of stitching 512 each include the stitching pattern 508 as shown in FIGS. 6A and 6B. In this configuration, first row of stitching 510 and second row of stitching 512 can be tightened to provide a compression force to the respective edges 320, 322 of implantable adjunct 300.

Referring again to FIG. 7, when tightened, first surface segments 502 and opposing surface segments 504 (as shown in FIGS. 6A and 6B) of both first row of stitching 510 and second row of stitching 512 are pulled together to compress implantable adjunct 300 at edges 320, 322. In this manner, edges 320, 322 are pre-compressed before insertion of a staple through implantable adjunct 300. The forces at edges 320, 322, generates an edge thickness 514 at each of edges 320, 322 for implantable adjunct 300 that are thinner than a center thickness 516 thereof. This provides rounded edges for implantable adjunct 300 that eases insertion of implantable adjunct 300 into first jaw frame 204, as shown in FIG. 1. A term of art in this sense is that the positive tension at the respective edges 320, 322 can assist with the aperture, i.e., a shape that aids the ability to place tissue between the first jaw frame 204 and anvil 210.

FIG. 8 shows an example double needle 600 that can be employed with a double-needle sewing machine to provide first row of stitching 510 and second row of stitching 512 as shown in FIG. 7, by way of example. Double needle 600 includes a first needle 602 and a second needle 604 that are spaced apart by a needle separation width 608. Double needle 600 can be used to provide first row of stitching 510 and the second row of stitching 512 that run parallel to each other to provide a consistent stich separation width 520 as illustrated and described with respect to FIGS. 6A and 6B above.

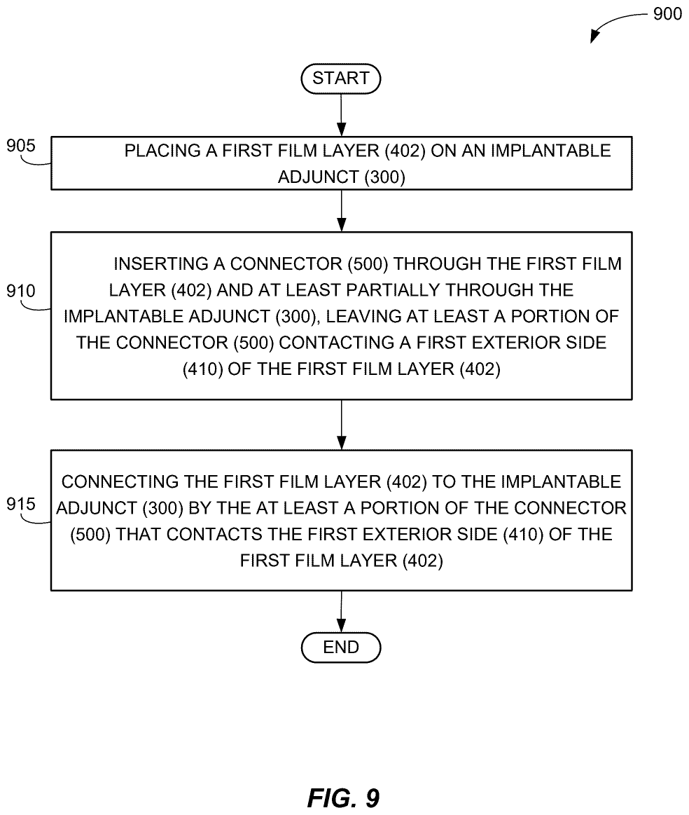

FIG. 9 is a flowchart of a method 900 for assembling an implantable adjunct 300. Method 900 will now be described with reference to FIGS. 1-9. In step 905, a first film layer 402 is placed on implantable adjunct 300. Although the method is described with respect to first film layer 402, it is to be understood that additional layers, such as second film layer 404, can be attached to and form a part of implantable adjunct 300.

Next, in step 910, connector 500, such as an absorbable thread, is inserted through first film layer 402. In one example, connector 500 is inserted at least partially through implantable adjunct 300. In another example, as shown in FIGS. 6A and 6B, connector 500 is inserted entirely through implantable adjunct 300 to provide first surface segments 502 and opposing surface segments 504 on opposing surfaces of implantable adjunct 300. In some examples, connector 500 can be a row of stitching that attaches first film layer 402 to implantable adjunct 300. Connector 500 can be inserted such that at least a portion of the connector 500 is left contacting first exterior side 410 of first film layer 402 to provide a force that attaches first film layer 402 to implantable adjunct 300.

In another example, as illustrated for example in FIG. 5, connector 500 can include first row of stitching 510 and second row of stitching 512. In this example, first row of stitching 510 and second row of stitching 512 are located at edges 320, 322, respectively, of implantable adjunct 300. First row of stitching 510 and second row of stitching 512 can be inserted, for example, using a double needle sewing machine employing double needle 600 as shown in FIG. 8, by way of example. Double needle 600 can be used with first needle 602 and second needle 604 that are separated by needle separation width 608. In one example, a fixture can be employed to align the implantable adjunct 300 and the first film layer 402 and feed both layers into the double needle sewing machine to attach the layers together at edges 320, 322. In this manner, first row stitching 510 and second row of stitching 512 can be inserted at stitch separation width 520 with first row of stitching 510 and second row of stitching 512 running parallel and along opposite edges 320, 322 of implantable adjunct 300.

As shown, for example, in FIGS. 5 and 6, connector 500 can be inserted in step 910 to leave a portion of connector 500, such as first surface segments 502 (first row of stitching 510) and second surface segments 522 (second row of stitching 512) that contact first exterior side 410 of first film layer 402. In one example, first row of stitching 510 and second row of stitching 512 are inserted using a double needing sewing machine including double needle 600 as shown in FIG. 8, such that first surface segments 502 and second surface segments 522 are aligned laterally with respect to each other along longitudinal axis 350 of implantable adjunct 300, as shown in FIG. 5.

In step 915, first film layer 402 is connected to implantable adjunct 300, for example, by first surface segments 502 of first row of stitching 510 and/or second surface segments 522 of second row of stitching 512. In the example shown in FIGS. 6A and 6B using stitching pattern 508, first row of stitching 510 and second row of stitching 512 can optionally be tightened at respective edges 320, 322 of implantable adjunct 300 to compress implantable adjunct 300 at edges 320, 322. In this manner, the forces at edges 320, 322, generate an edge thickness 514 at each of edges 320, 322 for implantable adjunct 300 that are thinner than a center thickness 516 thereof.

Examples of the present disclosure can be implemented by any of the following numbered clauses:
Clause 1: An implantable adjunct (300) configured to be detachably adhered to a deck (108) of a staple cartridge (100), the implantable adjunct (300) comprising: a deck-facing surface (316); an external surface (318); a first film layer (402) contacting the deck-facing surface (316) and comprising a first exterior side (410) facing away from the implantable adjunct (300); and a connector (500) in contact with the first exterior side (410), the connector (500) providing a force on the first exterior side (410) thereby attaching the first film layer (402) to the deck-facing surface (316).
Clause 2: The implantable adjunct (300) of Clause 1, wherein the connector (500) extends at least partially through the implantable adjunct (300).
Clause 3: The implantable adjunct (300) of Clause 1 or 2, wherein the connector (500) is a thread extending through the first film layer (402).
Clause 4: The implantable adjunct (300) of Clause 3, wherein a first surface segment (502) of the thread contacts the first exterior side (410) to provide the force, and wherein the thread comprises transverse segments (506) positioned at each side of the first surface segment (502), the transverse segments (506) extending at least partially through the implantable adjunct (300).
Clause 5: The implantable adjunct (300) of any one of the preceding Clauses, wherein the connector (500) comprises a first row of stitching (510) comprising a first plurality of surface segments (502), the first plurality of surface segments (502) providing the force on the first exterior side (410) of the first film layer (402).
Clause 6: The implantable adjunct (300) of Clause 5, wherein the first row of stitching (510) is positioned proximate a first edge (320) of the implantable adjunct (300).
Clause 7: The implantable adjunct (300) of Clause 5 or 6, wherein the connector (500) comprises a second row of stitching (512) comprising a second plurality of surface segments (522), the second plurality of surface segments (522) providing the force on the first exterior side (410) of the first film layer (402).
Clause 8: The implantable adjunct (300) of Clause 7, wherein the implantable adjunct comprises a second edge (322), the first row of stitching (510) and the second row of stitching (512) run parallel to each other and are proximate opposite edges (320, 322) of the implantable adjunct (300).
Clause 9: The implantable adjunct (300) of Clause 8, wherein the first plurality of surface segments (502) and the second plurality of surface segments (522) are aligned laterally with respect to each other along a longitudinal axis (350) of the implantable adjunct (300).
Clause 10: The implantable adjunct (300) of Clause 8 or 9, wherein an edge thickness (514) of the implantable adjunct (300) is thinner than a center thickness (516) responsive to the force provided by the first row of stitching (510) and the second row of stitching (512) pre-compressing the respective edges (320, 322).
Clause 11: The implantable adjunct (300) of any one of Clauses 3 to 10, wherein the thread comprises an absorbable material.
Clause 12: The implantable adjunct (300) of any one of the preceding Clauses, further comprising, a second film layer (404) contacting the external surface (318) and comprising a second exterior side (414), the connector (500) providing a force on the second exterior side (414) thereby attaching the second film layer (404) to the external surface (318).
Clause 13: The implantable adjunct (300) of any one of the preceding Clauses, further comprising the staple cartridge (100).
Clause 14: The implantable adjunct (300) of any one of the preceding Clauses, wherein the implantable adjunct (300) is adhered to the deck (108) by an attachment material (302) positioned between the first film layer (402) and the deck (108).
Clause 15: A method for assembling an implantable adjunct (300), the method comprising: placing a first film layer (402) on the implantable adjunct (300); inserting a connector (500) through the first film layer (402) and at least partially through the implantable adjunct (300), leaving at least a portion of the connector (500) contacting a first exterior side (410) of the first film layer (402); and connecting the first film layer (402) to the implantable adjunct (300) by the at least a portion of the connector (500) that contacts the first exterior side (410) of the first film layer (402).
Clause 16: The method of Clause 15, wherein: the connector (500) comprises thread; the step of inserting the connector (500) through the first film layer (402) and at least partially through the implantable adjunct (300) comprises creating a first row of stitching (510) along the first exterior side (410) of the first film layer (402); and the at least a portion of the connector (500) that contacts the first exterior side (410) is a first plurality of surface segments (502) of the thread.
Clause 17: The method of Clause 16, wherein: the step of inserting the connector (500) through the first film layer (402) and at least partially through the implantable adjunct (300) comprises creating a second row of stitching (512) along the first exterior side (410) of the first film layer (402); and the at least a portion of the connector (500) that contacts the first exterior side (410) is a second plurality of surface segments (522) of the thread.
Clause 18: The method of Clause 17, wherein the first row of stitching (510) and the second row of stitching (512) run parallel and along opposite edges (320, 322) of the implantable adjunct (300).
Clause 19: The method of any one of Clauses 16 to 18, wherein the first row of stitching (510) and the second row of stitching (512) are applied using a double needle sewing machine.
Clause 20: A system comprising: an implantable adjunct (300) comprising: a first absorbable material; and a film layer (402) attached to the implantable adjunct (300) by a first row of stitching (510) and a second row of stitching (512) that run parallel and proximate opposite edges (320, 322) of the implantable adjunct (300), the first row of stitching (510) and the second row of stitching (512) comprising a second absorbable material; a staple cartridge (100) comprising a deck (108); and an attachment material (302) attaching the implantable adjunct (300) to the deck (108).

In describing example embodiments, terminology has been resorted to for the sake of clarity. As a result, not all possible combinations have been listed, and such variants are often apparent to those of skill in the art and are intended to be within the scope of the claims which follow. It is intended that each term contemplates its broadest meaning as understood by those skilled in the art and includes all technical equivalents that operate in a similar manner to accomplish a similar purpose without departing from the scope and spirit of the invention. It is also to be understood that the mention of one or more steps of a method does not preclude the presence of additional method steps or intervening method steps between those steps expressly identified. Similarly, some steps of a method can be performed in a different order than those described herein without departing from the scope of the disclosed technology.

1-15. (canceled)

16. An implantable adjunct configured to be detachably adhered to a deck of a staple cartridge, the implantable adjunct comprising:
a deck-facing surface;
an external surface;
a first film layer contacting the deck-facing surface and comprising a first exterior side facing away from the implantable adjunct; and
a connector in contact with the first exterior side, the connector providing a force on the first exterior side thereby attaching the first film layer to the deck-facing surface.

17. The implantable adjunct of claim 16, wherein the connector extends at least partially through the implantable adjunct.

18. The implantable adjunct of claim 16, wherein the connector is a thread extending through the first film layer.

19. The implantable adjunct of claim 18, wherein a first surface segment of the thread contacts the deck-facing surface to provide the force, and wherein the thread comprises transverse segments positioned at each side of the first surface segment, the transverse segments extending at least partially through the implantable adjunct.

20. The implantable adjunct of claim 16, wherein the connector comprises a first row of stitching comprising a first plurality of surface segments, the first plurality of surface segments providing the force on the first exterior side of the first film layer.

21. The implantable adjunct of claim 20, wherein the first row of stitching is positioned proximate a first edge of the implantable adjunct.

22. The implantable adjunct of claim 20, wherein the connector comprises a second row of stitching comprising a second plurality of surface segments, the second plurality of surface segments providing the force on the first exterior side of the first film layer.

23. The implantable adjunct of claim 22, wherein the implantable adjunct comprises a second edge, the first row of stitching and the second row of stitching run parallel to each other and are proximate opposite edges of the implantable adjunct.

24. The implantable adjunct of claim 23, wherein the first plurality of surface segments and the second plurality of surface segments are aligned laterally with respect to each along a longitudinal axis of the implantable adjunct.

25. The implantable adjunct of claim 23, wherein an edge thickness of the implantable adjunct is thinner than a center thickness responsive to the force provided by the first row of stitching and the second row of stitching pre-compressing the respective edges.

26. The implantable adjunct of claim 18, wherein the thread comprises an absorbable material.

27. The implantable adjunct of claim 16, further comprising, a second film layer contacting the external surface and comprising a second exterior side, the connector providing a force on the second exterior side thereby attaching the second film layer to the external surface.

28. The implantable adjunct of claim 16, further comprising the staple cartridge.

29. The implantable adjunct of claim 16, wherein the implantable adjunct is adhered to the deck by an attachment material positioned between the first film layer and the deck.

30. A method for assembling an implantable adjunct, the method comprising:
placing a first film layer on the implantable adjunct;
inserting a connector through the first film layer and at least partially through the implantable adjunct, leaving at least a portion of the connector contacting a first exterior side of the first film layer; and
connecting the first film layer to the implantable adjunct by the at least a portion of the connector that contacts the first exterior side of the first film layer.

31. The method of claim 30, wherein:
the connector comprises thread;
the step of inserting the connector through the first film layer and at least partially through the implantable adjunct comprises creating a first row of stitching along the first exterior side of the first film layer; and
the at least a portion of the connector that contacts the first exterior side is a first plurality of surface segments of the thread.

32. The method of claim 31, wherein:
the step of inserting the connector through the first film layer and at least partially through the implantable adjunct comprises creating a second row of stitching along the first exterior side of the first film layer; and
the at least a portion of the connector that contacts the first exterior side is a second plurality of surface segments of the thread.

33. The method of claim 32, wherein the first row of stitching and the second row of stitching run parallel and along opposite edges of the implantable adjunct.

34. The method of any one of claims 31 to 33, wherein the first row of stitching and the second row of stitching are applied using a double needle sewing machine.

35. A system comprising:
an implantable adjunct comprising:
a first absorbable material; and
a film layer attached to the implantable adjunct by a first row of stitching and a second row of stitching that run parallel and proximate opposite edges of the implantable adjunct, the first row of stitching and the second row of stitching comprising a second absorbable material;
a staple cartridge comprising a deck; and
an attachment material attaching the implantable adjunct to the deck.
